SEQUENCE LISTING (1 sequence)

SEQ ID NO: 1 - protein, 20 aa, Scylla paramamosain
GKKKKRNMMKTKEPGIIFFF

(19) United States
(12) Patent Application Publication
(10) Pub. No.: US 2024/0138406 A1
(43) Pub. Date: May 2, 2024

(54) ANTIMICROBIAL PEPTIDE SCYAMPCIN44-63 AND APPLICATION THEREOF

(71) Applicant: Xiamen University, Xiamen (CN)

(72) Inventors: Kejian WANG, Xiamen (CN); Ying ZHOU, Xiamen (CN); Fangyi CHEN, Xiamen (CN)

(21) Appl. No.: 18/398,278

(22) Filed: Dec. 28, 2023

Related U.S. Application Data
(63) Continuation of application No. PCT/CN2022/100777, filed on Jun. 23, 2022.

(30) Foreign Application Priority Data
Jun. 28, 2021 (CN) 202110722932.7

(51) Int. Cl. A01N 37/46; A01P 1/00; A01P 3/00; C07K 14/435

(52) U.S. Cl. CPC A01N 37/46; A01P 1/00; A01P 3/00; C07K 14/43509

(57) ABSTRACT

An antimicrobial peptide Scyampcin₄₄₋₆₃, a formula of the antimicrobial peptide Scyampcin₄₄₋₆₃ is C₁₁₃H₁₈₈N₃₀O₂₄S₂, and an amino acid sequence of the antimicrobial peptide Scyampcin₄₄₋₆₃ is SEQ ID NO: 01 with the C-terminus being amidated.

### RELATED APPLICATIONS

This application is a continuation of International patent application PCT/CN2022/100777, filed on Jun. 23, 2022, which claims priority to Chinese patent application 202110722932.7, filed on Jun. 28, 2021. International patent application PCT/CN2022/100777 and Chinese patent application 202110722932.7 are incorporated herein by reference.

### REFERENCE TO AN ELECTRONIC SEQUENCE LISTING

The contents of the electronic sequence listing (SequenceListing.xml; Size: 1,908 bytes; and Date of Creation: Dec. 28, 2023) is herein incorporated by reference.

### FIELD OF THE DISCLOSURE

The present disclosure relates to the technical field of marine molecular biotechnology, and in particular relates to a novel antimicrobial peptide named Scyampcin₄₄₋₆₃, which has broad-spectrum antimicrobial activity, and an application thereof.

### BACKGROUND OF THE DISCLOSURE

Tremendous multi drug-resistant (MDR), extensive drug-resistant (XDR), and pan drug-resistant (PDR) bacteria have emerged in clinics due to improper use of antibiotics, which causes great challenges to clinical therapy, and even trapped in unavailable drugs. Resistant bacteria have spread around the world. Every year, more than 700,000 people die from infections of the resistant bacteria. The World Health Organization predicts that the number will reach 10 million in 2050 without positive action. In 2017, the World Health Organization first published a list of “Priority pathogens” with antibiotic resistant and strengthened 12 categories of bacteria greatly threatening human health. The list of critical-priority 1 pathogens includes carbapenem-resistant Acinetobacter baumannii, carbapenem resistant Pseudomonas aeruginosa, and carbapenem-resistant/generating extended-spectrum β-lactamases Enterobacteriaceae bacteria (ESBL). Vancomycin-resistant Enterococcus faecium and methicillin-resistant/vancomycin-intermediate resistant Staphylococcus aureus are in the list of high-priority 2 pathogens. Exploring novel antibiotics to replace traditional antibiotics is extremely urgent due to a serious situation of antibiotic-resistant bacteria.

Invasive fungal infections are increasing as an expanding population of immune deficiencies. Invasive fungal infections caused by Candida albicans, Cryptococcus neoformans, Aspergillus fumigatus, Histoplasma capsulatum, and etc. take away 1.5 million lives annually, which seriously threatens human health. Nowadays, the available antifungal drugs are relatively limited and mainly include azoles (with cross drug-resistance), polyenes (such as amphotericin B, with severe nephrotoxicity), echinocandins (with short half-life), and fluorocytosine (with side effects, such as nausea, vomiting, diarrhea, and bone marrow suppression). In addition to the aforementioned problems, antifungal drug-resistance of fungi has become increasingly deterioration in recent years, and drug resistance of azoles, polyenes, echinocandins, and fluorocytosine keeps emerging. The most typical azoles-resistant problem is induced by wide use of azoles in agriculture. In addition, invasive fungal infections have more than a 20% lethal rate. Exploring new antifungal drugs is also urgent.

Antimicrobial peptides (AMPs), also known as host defense peptides, are usually an amphiphilic cationic polypeptide. The AMPs have broad-spectrum antibacterial, antifungal, antiviral, antiparasitic, anti-tumor, and etc. activities, which widely exist in organisms, such as animals, plants, and microorganisms. As an effector molecule of innate immunity, the AMPs have diverse antimicrobial mechanisms, multiple targets, broad antimicrobial spectrum, rapid microbicide, and are less prone to produce drug resistance than the traditional antibiotics. In addition, the AMPs also promote wound healing and possess anti-inflammation and immune regulation. Thus, the AMPs are considered a good substitute for natural antibiotics. Natural peptides, synthetic peptides, or modified peptides have entered clinical trials for treatment of fungal or bacterial infections. Discovering and developing novel AMPs, seeking effective alternatives of antibiotics to reduce and alleviate clinical drug resistance are becoming extremely important since the AMPs has potential clinical application.

### BRIEF SUMMARY OF THE DISCLOSURE

A first objective of the present disclosure is to provide an antimicrobial peptide Scyampcin₄₄₋₆₃.

A second objective of the present disclosure is to provide an application of the antimicrobial peptide Scyampcin₄₄₋₆₃.

A first technical solution of the present disclosure is as follows:

An antimicrobial peptide Scyampcin₄₄₋₆₃, wherein an amino acid sequence of the antimicrobial peptide Scyampcin₄₄₋₆₃ is SEQ ID NO: 01.

The antimicrobial peptide of the present disclosure is named Scyampcin₄₄₋₆₃ with broad-spectrum antimicrobial activity, which derived from Scylla paramamosain, and a formula is C₁₁₃H₁₈₈N₃₀O₂₄S₂.

A second technical solution of the present disclosure is as follows:

An application of the antimicrobial peptide Scyampcin₄₄₋₆₃ comprises preparing an antibacterial composition by diluting the antimicrobial peptide Scyampcin₄₄₋₆₃ in water.

In a preferred embodiment of the present disclosure, the antibacterial composition has inhibitory and bactericidal effects on at least one of Staphylococcus aureus, Enterococcus faecium, Listeria monocytogenes, Bacillus cereus, Enterococcus faecalis, Shigella flexneri, Acinetobacter baumannii, Escherichia coli, Pseudomonas aeruginosa, Klebsiella pneumoniae, or Enterobacter cloacae.

A third technical solution of the present disclosure is as follows:

An antibacterial composition, wherein an effective component of the antibacterial composition comprises the antimicrobial peptide Scyampcin₄₄₋₆₃.

In a preferred embodiment of the present disclosure, the effective component of the antibacterial composition is the antimicrobial peptide Scyampcin₄₄₋₆₃.

A fourth technical solution of the present disclosure is as follows:

An application of the antimicrobial peptide Scyampcin₄₄₋₆₃ comprises preparing an antifungal composition by diluting the antimicrobial peptide Scyampcin₄₄₋₆₃ in water.

In a preferred embodiment of the present disclosure, the antifungal composition has inhibitory and bactericidal effects on at least one of yeast fungi or filamentous fungi.

Further preferably, the yeast fungi are Cryptococcus neoformans or Candida albicans, and the filamentous fungi are Fusarium solani or Fusarium graminearum.

A fifth technical solution of the present disclosure is as follows:

An antifungal composition, wherein an effective component of the antifungal composition comprises the antimicrobial peptide Scyampcin₄₄₋₆₃.

In a preferred embodiment of the present disclosure, the effective component of the antifungal composition is the antimicrobial peptide Scyampcin₄₄₋₆₃.

The present disclosure has the following advantages:

1. The broad-spectrum antimicrobial peptide Scyampcin₄₄₋₆₃ derived from Scylla paramamosain in the present disclosure consists of 20 amino acids (e.g., with a C-terminus being amidated), and a molecular weight is 2428.03 Dalton. The broad-spectrum antimicrobial peptide Scyampcin₄₄₋₆₃ comprises 6 lysines, 1 arginine, and 1 glycine. HeliQuest predicted that the broad-spectrum antimicrobial peptide Scyampcin₄₄₋₆₃ is charged +6 and has hydrophobicity of 0.211. The broad-spectrum antimicrobial peptide Scyampcin₄₄₋₆₃ is a cationic short peptide with good water solubility, potent and broad-spectrum antimicrobial activity, is safe, and has broad application prospects. 2. The broad-spectrum antimicrobial peptide Scyampcin₄₄₋₆₃ derived from Scylla paramamosain in the present disclosure has potent antimicrobial activity against clinically drug-resistant bacteria and fungi and has no cytotoxicity to human embryonic kidney 293T (HEK-293T) and epithelioma papulosum cyprini (EPC) cells.

### BRIEF DESCRIPTION OF THE DRAWING

FIGS. 1A and 1B respectively show time-killing kinetics of a broad-spectrum antimicrobial peptide, Scyampcin₄₄₋₆₃, derived from Scylla paramamosain against Acinetobacter baumannii (CGMCC 1.6769) and Staphylococcus aureus (CGMCC 1.2465) in Embodiment 1 of the present disclosure.

FIGS. 2A, 2B, 2C, 2D, 2E, and 2F show morphological and structural changes of Staphylococcus aureus (CGMCC 1.2465), Escherichia coli (CGMCC 1.2389), and Candida albicans (CGMCC 2.2411) caused by a treatment of the Scyampcin₄₄₋₆₃ in Embodiment 1 of the present disclosure. FIG. 2A shows a control group of Staphylococcus aureus, FIG. 2B shows Staphylococcus aureus treated with 6 μMol/L (μM) Scyampcin₄₄₋₆₃, FIG. 2C shows a control group of Escherichia coli, FIG. 2D shows Escherichia coli treated with 3 μM Scyampcin₄₄₋₆₃, FIG. 2E shows a control group of Candida albicans, and FIG. 2F shows Candida albicans treated with 6 μM Scyampcin₄₄₋₆₃.

FIGS. 3A and 3B show cytotoxicity test results of the Scyampcin₄₄₋₆₃ derived from Scylla paramamosain against HEK-293T cells and EPC cells in Embodiment 1 of the present disclosure. The abscissa is a concentration of the Scyampcin₄₄₋₆₃ (μM), and the ordinate is cell viability (%).

### DETAILED DESCRIPTION OF THE EMBODIMENTS

The present disclosure will be further described in combination with the accompanying drawings and embodiments.

### Embodiment 1

An amino acid sequence of a broad-spectrum antimicrobial peptide Scyampcin₄₄₋₆₃ derived from Scylla paramamosain (i.e., antimicrobial peptide Scyampcin₄₄₋₆₃ or Scyampcin₄₄₋₆₃) is SEQ ID NO: 01 (GKKKKRNMMKTKEPGIIFFF-NH₂) with a C-terminus that is amidated (—NH₂), that is, phenylalanine at position 20 is modified by —NH₂.

In this embodiment, the Scyampcin₄₄₋₆₃ with a purity of more than 95% is chemically synthesized by Genscript (Nanjing, China) by a solid-phase synthesis method, and analysis information, such as polypeptide molecular weight and high performance liquid chromatography (HPLC), is provided. HeliQuest is used to predict charge and hydrophobicity of the Scyampcin₄₄₋₆₃, and ProtParam is used to predict the remaining physicochemical parameters. Physicochemical parameters of the Scyampcin₄₄₋₆₃ are shown in Table 1.

TABLE 1
Physicochemical parameters of the Scyampcin₄₄₋₆₃

| Physicochemical parameters | Scyampcin₄₄₋₆₃ |
|---|---|
| Amino acid residues | 20 |
| Molecular weight | 2428.03 Dalton |
| Isoelectric point | 10.68 |
| Net charge | +6 |
| Hydrophobicity | 21.1% |

I. Assessment of a minimum bactericidal concentration (MBC) of the Scyampcin₄₄₋₆₃

1. The following strains are involved: Cryptococcus neoformans, Candida albicans, Fusarium solani, Fusarium graminearum, Staphylococcus aureus, Enterococcus faecium, Enterococcus faecalis, Listeria monocytogenes, Bacillus cereus, Escherichia coli, Acinetobacter baumannii, and Pseudomonas aeruginosa are purchased from the China General Microbiological Culture Collection Center (CGMCC); Clinical drug-resistant bacteria, including QZ19131, QZ18080, QZ19138, QZ20141, QZ18109, QZ18071, QZ18106, and QZ18103, are from Second Affiliated Hospital of Fujian Medical University (Fujian, China).

2. A specific method is as follows:
(1) The preserved bacteria strains are streaked on a nutrient broth (NB) plate, incubated at 37° C. while leaving to stand. Yeast fungi are streaked on a Yeast Extract Peptone Dextrose (YPD) plate, filamentous fungi are streaked on a Potato Dextrose Agar (PDA) plate, and the yeast fungi and the filamentous fungi are incubated at 28° C. while leaving to stand.
(2) When colonies of the strains grow to a proper size, the colonies of the strains are picked and transferred to an NB liquid medium, shaken at 37° C., and incubated at 180 revolutions per minute (rpm) until reaching logarithmic growth phase. Colonies of the yeast fungi are picked and transferred to a YPD liquid medium, shaken at 28° C., and incubated at 230 rpm until reaching a logarithmic growth phase.
(3) The bacteria and the yeast fungi are collected by centrifugation. The bacteria are diluted with Mueller-Hinton (MH) liquid and adjusted to about 5*10⁵ cfu/mL. The yeast fungi are diluted with Roswell Park Memorial Institute (RPMI)1640+0.165 Mol/L (M) 3-morpholinopropane-1-sulfonic acid (MOPS)+0.2% glucose and adjusted to about 5*10⁴ cfu/mL. Spores of the filamentous fungi are collected, counted, and adjusted to 5*10⁴ cfu/mL using a medium of RPMI1640+0.165 M MOPS.
(4) Powder of the synthesized Scyampcin₄₄₋₆₃ is dissolved in sterile Milli-Q® water, filtered with a 0.22 μm filter membrane, and diluted until a concentration of the synthesized Scyampcin₄₄₋₆₃ is 6 μM, 12 μM, 24 μM, 48 μM, 96 μM, or 192 μM.

(5) A blank control group, a negative control group, and an experimental group of each of the bacteria, the yeast fungi, and the filamentous fungi are seeded on 96-well cell culture plates, and each of the blank control group, the negative control group, and the experimental group has three parallel samples.

The blank control group a: 50 μL of the Spampcin₄₄₋₆₃ with 50 μL of a corresponding culture medium for diluting the bacteria, the yeast fungi, and the filamentous fungi;

The negative control group b: 50 μL of the sterilized MilliQ® water and 50 μL suspension of the bacteria, the yeast fungi, or the spores of the filamentous fungi;

The experimental group c: 50 μL of the Spampcin₄₄₋₆₃ and 50 μL suspension of the bacteria, the yeast fungi, or the spores of the filamentous fungi.

The 96-well cell culture plates are placed at 37° C. or 28° C., incubated for 24 hours, and mixed thoroughly to obtain a co-incubated mixed solution (e.g., co-incubated suspension). 2 μL of the co-incubated mixed solution is spread on the NB plate or the YPD plate, and incubated at 37° C. or 28° C. Results of the MBC are observed and recorded.

3. Observation results of the MBC of the broad-spectrum antimicrobial peptide Scyampcin₄₄₋₆₃ of Scylla paramamosain are shown in Table 2.

TABLE 2
Antimicrobial activity of the Scyampcin₄₄₋₆₃

| Microorganism | NO. | MBC (μM) |
|---|---|---|
| Gram-positive bacterium | | |
| Staphylococcus aureus | CGMCC 1.2465 | 3-6 |
| Enterococcus faecium | CGMCC 1.131 | 0-3 |
| Listeria monocytogenes | CGMCC 1.10753 | 0-3 |
| Bacillus cereus | CGMCC 1.3760 | 3-6 |
| Staphylococcus aureus | QZ19131 | 3-6 |
| Enterococcus faecium | QZ18080 | 0-3 |
| Enterococcus faecalis | QZ19138 | 3-6 |
| Gram-negative bacterium | | |
| Shigella flexneri | CGMCC 1.1868 | 0-3 |
| Acinetobacter baumannii | CGMCC 1.6769 | 0-3 |
| Escherichia coli | CGMCC 1.2389 | 0-3 |
| Pseudomonas aeruginosa | CGMCC 1.1785 | 6-12 |
| Acinetobacter baumannii | QZ20141 | 3-6 |
| Escherichia coli | QZ18109 | 0-3 |
| Pseudomonas aeruginosa | QZ18071 | 6-12 |
| Klebsiella pneumoniae | QZ18106 | 3-6 |
| Enterobacter cloacae | QZ18103 | 3-6 |
| Yeast fungi | | |
| Cryptococcus neoformans | CGMCC 2.1563 | 0-3 |
| Candida albicans | CGMCC 2.2411 | 3-6 |
| Filamentous fungi | | |
| Fusarium graminearum | CGMCC 3.4521 | 3-6 |
| Fusarium solani | CGMCC 3.584 | 3-6 |

II. Time-killing kinetics of the Scyampcin₄₄₋₆₃

1. The following strains are involved: Acinetobacter baumannii (CGMCC 1.6769) and Staphylococcus aureus (CGMCC 1.2465).

2. A specific method is as follows:

Time-killing kinetics of the Scyampcin₄₄₋₆₃ are performed in the same way as the assay of the MBC of the Scyampcin₄₄₋₆₃. After the Scyampcin₄₄₋₆₃ and the stains are incubated for a preset time to obtain a co-incubated mixed solution (e.g., a co-incubated suspension), 10 μL of the co-incubated mixed solution taken at different time points are added into 490 μL of phosphate buffered saline (PBS) and sufficiently mixed to even to obtain gradient diluents, and 50 μL of each of the gradient diluents is spread onto a first NB plate. If the colonies of the first NB plate are less than 50, 25 μL of the co-incubated mixed solution is spread onto a second NB plate directly. After incubation at 37° C. for 12 hours while leaving to stand, the colonies of the second NB plate are counted.

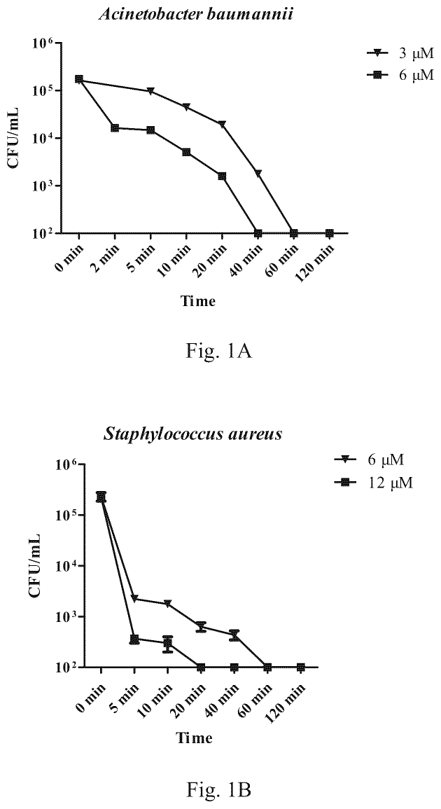

3. The time-killing kinetic of the Scyampcin₄₄₋₆₃ against Acinetobacter baumannii CGMCC 1.6769 and Staphylococcus aureus CGMCC 1.2465 are shown in FIGS. 1A and 1B.

III. Morphological and structural change of bacteria or fungi after being treated with the Scyampcin₄₄₋₆₃ are observed by a scanning electron microscope

1. The following strains are involved: Staphylococcus aureus CGMCC 1.2465, Escherichia coli CGMCC 1.2389, and Candida albicans CGMCC 2.2411.

2. A specific experimental method is as follows:
(1) Samples for the scanning electron microscope are prepared

The strains are activated, 3-5 colonies are randomly selected and transferred to corresponding nutrient broth liquid mediums, and shaken to a logarithmic growth phase. An optical density (OD) is measured, a first supernatant is removed by centrifugation, the strains are resuspended in an MH medium, and the OD is adjusted to 0.2 to obtain a strain solution. 500 μL of the strain solution are incubated with an equal volume of the Scyampcin₄₄₋₆₃ at 37° C. for 30 minutes (while the fungi are incubated in a YPD liquid medium to the logarithmic growth phase, the OD is measured, the first supernatant is removed by centrifugation, the fungi are resuspended in RPMI1640+0.165 M MOPS+0.2% glucose to obtain a fungal solution (e.g., a fungal suspension), and the OD is adjusted to 0.5. 500 μL of the fungal suspension is incubated with an equal volume of the Scyampcin₄₄₋₆₃ at 37° C. for 60 minutes). A second supernatant is removed by centrifugation, and microbe is harvested after washing with PBS.

(2) Fixation, washing, and adhesion:

The harvested microbe is suspended in 300 μL of 2.5% glutaraldehyde and fixed at 4° C. for 1.5 hours or more. The fixed microbe is washed with PBS for three times (20 minutes/time) to prepare a high concentration suspension, and the high concentration suspension is transferred onto glass slides to be adhered for 30 minutes to obtain samples. After adhesion, the samples are dried with filter paper and dehydrated in graded ethanol.

(3) Dehydration

The dehydration is sequentially performed by using 30% ethanol for 5 minutes, 50% ethanol for 5 minutes, 70% ethanol for 10 minutes, and is optionally refrigerated at 4° C. overnight. The aforementioned operations are performed in an ice box. The dehydration is sequentially performed using 80% ethanol for 10 minutes, 95% ethanol for 15 minutes, and 100% ethanol for 15 minutes twice.

(4) Then all of the samples are critical point dried, coated with gold particles (10 mA current for 60 seconds), and photographed with a scanning electron microscope.

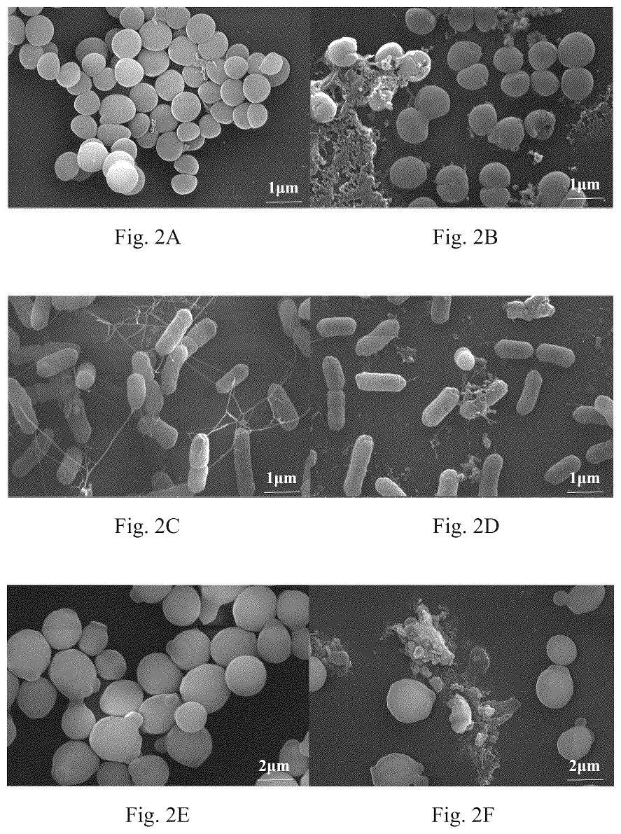

3. The morphological and structural change caused by the Scyampcin₄₄₋₆₃ of Scylla paramamosain against Staphylococcus aureus (CGMCC 1.2465), Escherichia coli (CGMCC 1.2389), and Candida albicans (CGMCC 2.2411) are shown in FIGS. 2A, 2B, 2C, 2D, 2E, and 2F.

IV. Cytotoxicity of the Scyampcin₄₄₋₆₃ is measured and evaluated by using 3-(4,5-dimethylthiazol-2-yl)-5-(3-carboxymethoxyphenyl)-2-(4-sulfophenyl)-2H-tetrazolium (MTS)

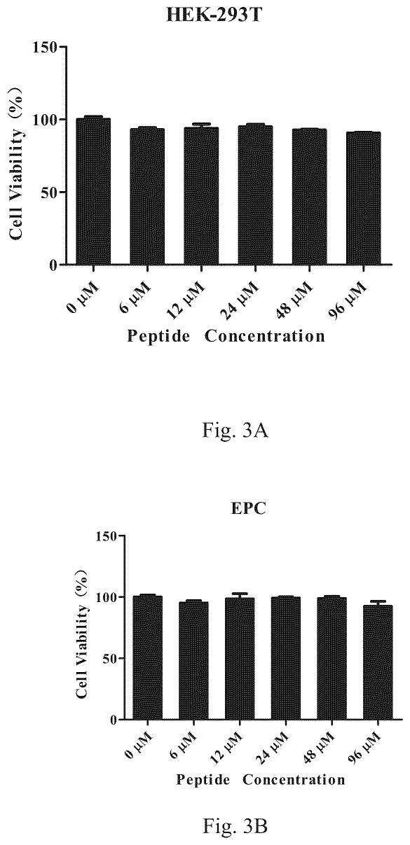

1. Human embryonic kidney 293T (HEK-293T) cells and epithelioma papulosum cyprini (EPC) cells are selected to assess the cytotoxicity of the Scyampcin₄₄₋₆₃. As shown in FIGS. 3A and 3B, an abscissa is a concentration of the Scyampcin₄₄₋₆₃ (μM), and an ordinate is a cell viability (%).

2. A specific method is as follows:

(1) The cells are respectively collected and diluted with DMEM+10% fetal bovine serum (FBS) to obtain a cell suspension with a final cell concentration of 10⁵ cells/mL, and 100 μL of the cell suspension is seeded in a 96 well cell culture plate. HEK-293T cells are incubated in a cell incubator with 5% CO₂ at 37° C., and EPC cells are incubated in a cell incubator with 5% CO₂ at 28° C.

(2) 50 μL of medium is carefully sucked out, fresh media with or without different concentrations of the Scyampcin₄₄₋₆₃ are respectively added to a corresponding well, put in a corresponding cell condition, and incubated for another 24 hours while leaving to stand.

(3) 20 μL of an MTS-Phenazinemethosulfate (PMS) mixed solution is added and incubated for 4 hours, absorbance of each well is measured at OD_{492 nm} in a microplate reader, and cell viabilities are calculated.

3. The results are shown in FIGS. 3A and 3B. The Scyampcin₄₄₋₆₃ has no cytotoxicity to the HEK-293T cells and the EPC cells.

The aforementioned embodiments are merely some embodiments of the present disclosure, and the scope of the disclosure is not limited thereto. Thus, it is intended that the present disclosure cover any modifications and variations of the presently presented embodiments provided they are made without departing from the structure, the shape, and the principle of the present disclosure.

What is claimed is:

1. An antimicrobial peptide Scyampcin₄₄₋₆₃, wherein:
an amino acid sequence of the antimicrobial peptide Scyampcin₄₄₋₆₃ is SEQ ID NO: 01; or
the amino acid sequence of the antimicrobial peptide Scyampcin₄₄₋₆₃ is the sequence shown in the SEQ ID NO: 01 with at least one amino acid residue replaced, deleted, or inserted; or
the amino acid sequence of the antimicrobial peptide Scyampcin₄₄₋₆₃ has more than 80% homology with the sequence shown in the SEQ ID NO: 01.

2. A nucleic acid encoding the antimicrobial peptide Scyampcin₄₄₋₆₃ according to claim 1.

3. At least one of the following biomaterials:
1) an expression cassette comprising the nucleic acid according to claim 2;
2) a recombinant carrier comprising the nucleic acid according to claim 2;
3) a recombinant bacterium or a transgenic cell line comprising the nucleic acid according to claim 2; or
4) nanoparticles comprising the recombinant carrier.

4. An application of the antimicrobial peptide Scyampcin₄₄₋₆₃ according to claim 1, comprising:
preparing an antibacterial composition by diluting the antimicrobial peptide Scyampcin₄₄₋₆₃ in water.

5. The application according to claim 4, wherein:
the antibacterial composition has inhibitory and bactericidal effects on at least one of Staphylococcus aureus, Enterococcus faecium, Listeria monocytogenes, Bacillus cereus, Enterococcus faecalis, Shigella flexneri, Acinetobacter baumannii, Escherichia coli, Pseudomonas aeruginosa, Klebsiella pneumoniae, or Enterobacter cloacae.

6. An application of the antimicrobial peptide Scyampcin₄₄₋₆₃ according to claim 1, comprising:
preparing an antifungal composition by diluting the antimicrobial peptide Scyampcin₄₄₋₆₃ in water.

7. The application according to claim 6, wherein:
the antifungal composition has inhibitory and bactericidal effects on at least one of yeast fungi or filamentous fungi.

8. The application according to claim 7, wherein:
the yeast fungi are Cryptococcus neoformans or Candida albicans, and
the filamentous fungi are Fusarium solani or Fusarium graminearum.

9. An antibacterial composition, wherein:
an effective component of the antibacterial composition comprises an antimicrobial peptide Scyampcin₄₄₋₆₃, and
an amino acid sequence of the antimicrobial peptide Scyampcin₄₄₋₆₃ is shown in SEQ ID NO: 01.

10. An antifungal composition, wherein:
an effective component of the antifungal composition comprises an antimicrobial peptide Scyampcin₄₄-63, and
an amino acid sequence of the antimicrobial peptide Scyampcin₄₄₋₆₃ is shown in SEQ ID NO: 01.

11. The antimicrobial peptide Scyampcin₄₄₋₆₃ according to claim 1, wherein the amino acid sequence of the antimicrobial peptide Scyampcin₄₄₋₆₃ has more than 85% homology with the sequence shown in the SEQ ID NO: 01.

12. The antimicrobial peptide Scyampcin₄₄₋₆₃ according to claim 1, wherein the amino acid sequence of the antimicrobial peptide Scyampcin₄₄₋₆₃ has more than 90% homology with the sequence shown in the SEQ ID NO: 01.

13. The antimicrobial peptide Scyampcin₄₄₋₆₃ according to claim 1, wherein the amino acid sequence of the antimicrobial peptide Scyampcin₄₄₋₆₃ has more than 99% homology with the sequence shown in the SEQ ID NO: 01.

14. The antimicrobial peptide Scyampcin₄₄₋₆₃ according to claim 1, wherein the sequence shown in the SEQ ID NO: 01 with at least one amino acid residue replaced, deleted, or inserted comprise SEQ ID NO: 01 with a C-terminus being is amidated.
